(19) United States
(12) Patent Application Publication
(10) Pub. No.: US 2018/0133419 A1
(43) Pub. Date: May 17, 2018

(54) MONITORING THE DEGRADATION OF A COMPONENT OF A PATIENT INTERFACE DEVICE

(71) Applicant: KONINKLIJKE PHILIPS N.V., EINDHOVEN (NL)

(72) Inventors: ROBERT WILLIAM BAIKO, PITTSBURGH, PA (US); DAVID W. SMITH, OAKMONT, PA (US)

(21) Appl. No.: 15/571,890

(22) PCT Filed: Apr. 25, 2016
(86) PCT No.: PCT/IB2016/052321

§ 371 Date: Nov. 6, 2017

Related U.S. Application Data
(60) Provisional application No. 62/158,038, filed on May 7, 2015.

(51) Int. Cl. A61M 16/00; A61M 16/06

(52) U.S. Cl. CPC A61M 16/0051; A61M 16/0066; A61M 16/024; A61M 16/06; A61M 2205/583; A61M 2205/15; A61M 2205/502; A61M 2205/3592; A61M 16/0003

(57) ABSTRACT

A pressure support system for delivering a flow of breathing gas to a patient includes a pressure generating base unit including a gas flow generator and a controller, a patient interface device for delivering the flow of breathing gas to the airway of the patient, and a degradation monitor apparatus including a control unit and an indicator apparatus. The controller is structured to generate data indicative of a degradation metric based on use of the pressure support system and to communicate the data indicative of the degradation metric to the degradation monitor apparatus. The control unit of the degradation monitor apparatus is structured to cause the indicator apparatus to generate a human perceptible indication based on the data indicative of the degradation metric when the degradation metric indicates that a component of the patient interface device demonstrates at least a threshold level of degradation.

### CROSS-REFERENCE TO RELATED APPLICATIONS

This patent application claims the priority benefit under 35 U.S.C. §119(e) of U.S. Provisional Application No. 62/158,038, filed on May 7, 2015, the contents of which are herein incorporated by reference.

### BACKGROUND OF THE INVENTION

### 1. Field of the Invention

The present invention pertains to systems for treating conditions, such as sleep disordered breathing, using positive airway pressure (PAP) therapy, and, in particular, to a system and method for monitoring the degradation of a component of a patient interface device of a pressure support system, and for alerting a user of the pressure support system that the component has degraded to the point where it should be replaced.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

Typically in the market today, insurance companies will periodically reimburse a person for a mask, cushion, headgear, and therapy device. The frequency of the reimbursement will often vary with the type of insurance or product. The problem with current masks is that there is no mechanism for indicating to the patient that the patient is getting less than optimal therapy because of a degradation of their therapy device (e.g., a degradation of a component of the patient interface device). Patients often will have a hard time benchmarking the performance of a mask on day one and comparing it to the same mask on day thirty. Often times a patient will complain that the mask is experiencing leaks and, rather than replacing the mask because it has degraded, the patient will tighten the mask until the leaks are gone, causing irritation and red marks down the line.

### SUMMARY OF THE INVENTION

In one embodiment, a pressure support system for delivering a flow of breathing gas to an airway of a patient is provided that includes a pressure generating base unit including a gas flow generator for generating the flow of breathing gas and a controller, a patient interface device for delivering the flow of breathing gas to the airway of the patient, and a degradation monitor apparatus including a control unit and an indicator apparatus. The controller is structured to generate data indicative of a degradation metric based on use of the pressure support system and to communicate the data indicative of the degradation metric to the degradation monitor apparatus. The control unit of the degradation monitor apparatus is structured to cause the indicator apparatus to generate a human perceptible indication based on the data indicative of the degradation metric when the degradation metric indicates that a component of the patient interface device demonstrates at least a threshold level of degradation.

In another embodiment, a method of monitoring degradation of a component of a patient interface device of a pressure support system structured to deliver a flow of breathing gas to an airway of a patient is provided. The method includes generating data indicative of a degradation metric in a pressure generating base unit of the pressure support system based on use of the pressure support system, communicating the data indicative of the degradation metric to a user interface apparatus, and generating from the user interface apparatus a human perceptible indication based on the data indicative of the degradation metric when the degradation metric indicates that the component of the patient interface device demonstrates at least a threshold level of degradation.

In yet another embodiment, a degradation monitor apparatus for monitoring degradation of a component of a patient interface device of a pressure support system structured to deliver a flow of breathing gas to an airway of a patient is provided. The degradation monitor apparatus includes a housing, an indicator apparatus supported by the housing, a wireless communications module within the housing, and a control unit within the housing, wherein the control unit is structured to (i) receive data indicative of a degradation metric generated by the pressure support system and based on use of the pressure support system through the wireless communications module, and (ii) cause the indicator apparatus to generate a human perceptible indication based on the data indicative of the degradation metric when the degradation metric indicates that a component of the patient interface device demonstrates at least a threshold level of degradation.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of an airway pressure support system according to one particular, non-limiting exemplary embodiment in which the present invention may be implemented;

FIG. 2 is a block diagram of a degradation monitor of the airway pressure support system of FIG. 1 according to the exemplary embodiment;

FIGS. 3A and 3B are top plan and side elevational views, respectively, of the degradation monitor of FIG. 2 according to one particular exemplary embodiment;

FIGS. 4A and 4B are top plan and side elevational views, respectively, of the degradation monitor of FIG. 2 according to another particular exemplary embodiment;

FIG. 5 is a schematic diagram of a headgear component of a patient interface device of the airway pressure support system of FIG. 1 according to one exemplary embodiment;

FIG. 6 is a schematic diagram of a pressure generating device base unit of the airway pressure support system of FIG. 1 according to another exemplary embodiment;

FIG. 7 is a flowchart illustrating a method of operation of the pressure support system of FIG. 1 wherein degradation of a component of a patient interface device thereof is monitored according to one exemplary embodiment; and

FIG. 8 is a flowchart illustrating a method of operation of the pressure support system of FIG. 1 wherein degradation of a component of a patient interface device thereof is monitored according to another exemplary embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

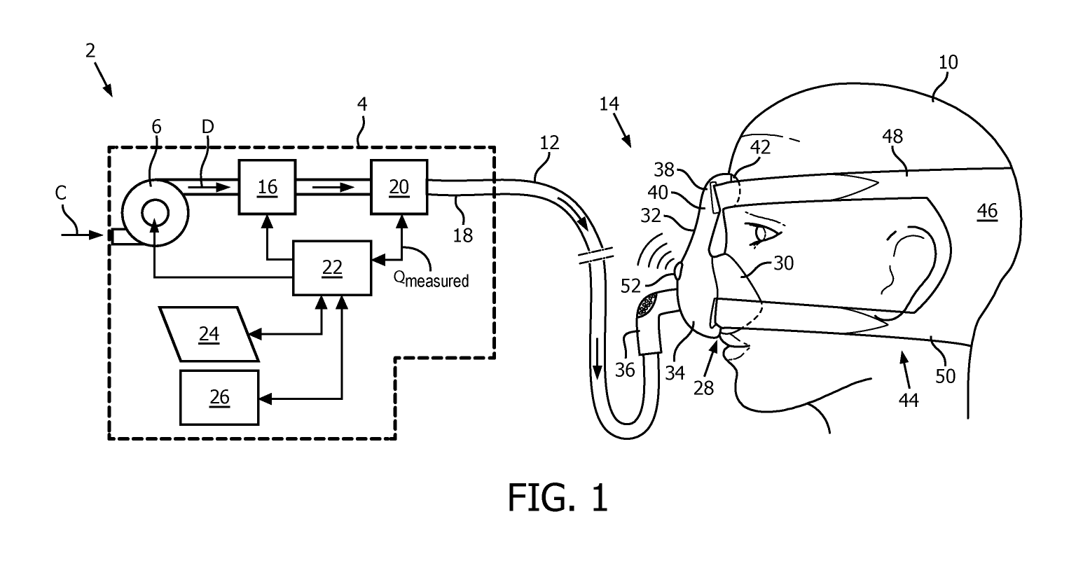

FIG. 1 is a schematic diagram of an airway pressure support system 2 according to one particular, non-limiting exemplary embodiment in which the present invention may be implemented. As described in detail herein, pressure support system 2 is provided with a mechanism for monitoring the degradation of a component of a patient interface device of pressure support system 2, and for alerting a user of pressure support system 2 that the component has degraded to the point where it should be replaced.

Referring to FIG. 1, airway pressure support system 2 includes a pressure generating device base unit 4 which houses a gas flow generator 6, such as a blower used in a conventional CPAP or bi-level pressure support device. Gas flow generator 6 receives breathing gas, generally indicated by arrow C, from the ambient atmosphere and generates a flow of breathing gas therefrom for delivery to an airway of a patient 10 at relatively higher and lower pressures, i.e., generally equal to or above ambient atmospheric pressure. In the exemplary embodiment, gas flow generator 6 is capable of providing a flow of breathing gas ranging in pressure from 3-30 cmH2O. The pressurized flow of breathing gas from gas flow generator 6, generally indicated by arrow D, is delivered via a delivery conduit 12 to a patient interface device 14 of any known construction, which is typically worn by or otherwise attached to patient 10 to communicate the flow of breathing gas to the airway of patient 10. Delivery conduit 12 and patient interface device 14 are typically collectively referred to as a patient circuit.

Pressure support system 2 shown in FIG. 1 is what is known as a single-limb system, meaning that the patient circuit includes only delivery conduit 12 connecting patient 10 to pressure support system 2. As such, an exhaust vent (described herein) is provided in in patient interface device 14 for venting exhaled gases from the system. It should be noted that the exhaust vent can be provided at other locations in addition to or instead of in patient interface device 14, such as in delivery conduit 12. It should also be understood that the exhaust vent can have a wide variety of configurations depending on the desired manner in which gas is to be vented from pressure support system 2.

The present invention also contemplates that pressure support system 2 can be a two-limb system, having a delivery conduit and an exhaust conduit connected to patient 10. In a two-limb system (also referred to as a dual-limb system), the exhaust conduit carries exhaust gas from patient 10 and includes an exhaust valve at the end distal from patient 10. The exhaust valve in such an embodiment is typically actively controlled to maintain a desired level or pressure in the system, which is commonly known as positive end expiratory pressure (PEEP).

Furthermore, in the illustrated exemplary embodiment shown in FIG. 1, patient interface device 14 is a nasal mask (the exemplary embodiment of which is described in detail herein). It is to be understood, however, that patient interface device 14 can include a nasal/oral mask, nasal pillows, a tracheal tube, an endotracheal tube, or any other device that provides a suitable gas flow communicating function. Also, for purposes of the present invention, the phrase “patient interface” can include delivery conduit 12 and any other structures that couple the source of pressurized breathing gas to patient 10.

In the illustrated embodiment, pressure support system 2 includes a pressure controller in the form of a valve 16 provided in internal delivery conduit 18 provided in pressure generating device base unit 4 of pressure support system 2. Valve 16 controls the pressure of the flow of breathing gas from gas flow generator 6 that is delivered to patient 10. For present purposes, gas flow generator 6 and valve 16 are collectively referred to as a pressure generating system because they act in concert to control the pressure and/or flow of gas delivered to patient 10. However, it should be apparent that other techniques for controlling the pressure of the gas delivered to patient 10, such as varying the blower speed of gas flow generator 6, either alone or in combination with a pressure control valve, are contemplated by the present invention. Thus, valve 16 is optional depending on the technique used to control the pressure of the flow of breathing gas delivered to patient 10. If valve 16 is eliminated, the pressure generating system corresponds to gas flow generator 6 alone, and the pressure of gas in the patient circuit is controlled, for example, by controlling the motor speed of gas flow generator 6.

Pressure support system 2 further includes a flow sensor 20 that measures the flow of the breathing gas within delivery conduit 20 and delivery conduit 12. In the particular embodiment shown in FIG. 1, flow sensor 20 is interposed in line with delivery conduits 20 and 12, most preferably downstream of valve 16. Flow sensor 20 generates a flow signal, Q_{MEASURED}, that is provided to a controller 22 and is used by controller 22 to determine the flow of gas at patient 10 (Q_{PATIENT}).

Techniques for calculating Q_{PATIENT} based on Q_{MEASURED} are well known, and take into consideration the pressure drop of the patient circuit, known leaks from the system, i.e., the intentional exhausting of gas from the circuit as described herein, and unknown (unintentional) leaks from the system, such as leaks at the mask/patient interface. The present invention contemplates using any known or hereafter developed technique for calculating total leak flow Q_{LEAK}, and using this determination in calculating Q_{PATIENT} based on Q_{MEASURED} (and for other purposes as described elsewhere herein). Examples of such techniques are taught by U.S. Pat. Nos. 5,148,802; 5,313,937; 5,433,193; 5,632,269; 5,803,065; 6,029,664; 6,539,940; 6,626,175; and 7,011,091, the contents of each of which are incorporated by reference into the present invention.

Of course, other techniques for measuring the respiratory flow of patient 10 are contemplated by the present invention, such as, without limitation, measuring the flow directly at patient 10 or at other locations along delivery conduit 12, measuring patient flow based on the operation of gas flow generator 6, and measuring patient flow using a flow sensor upstream of valve 16.

Controller 22 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for controlling the operation of pressure support system 2, including monitoring the degradation of a component of patient interface device 14 as described in greater detail herein.

An input/output device 24 is provided for setting various parameters used by airway pressure support system 2, as well as for displaying and outputting information and data to a user, such as a clinician or caregiver.

As seen in FIG. 1, pressure generating device base unit 4 in the exemplary embodiment also includes a short range wireless communications module 26 which is operatively coupled to controller 22. In the exemplary embodiment, short range wireless communications module 26 is a module that is structured and configured to enable pressure generating device base unit 22 to communicate with other, similarly equipped electronic devices (e.g., degradation monitor 52 described herein) over a short range wireless network. In the exemplary embodiment, short range wireless communications module 26 is a Bluetooth® module that is structured and configured to enable pressure generating device base unit 4 to communicate with other devices over an ad hoc Bluetooth® network. It will be appreciated, however, that other communication techniques/protocols, such as, without limitation, Wi-Fi, infrared light, near field communication (NFC), other RF based systems, a wired connection, etc.) may also be used within the scope of the present invention. In addition, short range wireless communications module 26 may be incorporated within pressure generating device base unit 4, or may be a module that is selectively connectable to pressure generating device base unit 4 via a USB port or other suitable connection.

In the illustrated, non-limiting exemplary embodiment of the present invention, airway pressure support system 2 essentially functions as a CPAP pressure support system, and, therefore, includes all of the capabilities necessary in such systems in order to provide appropriate CPAP pressure levels to patient 10. This includes receiving the necessary parameters, via input commands, signals, instructions or other information, for providing appropriate CPAP pressure, such as maximum and minimum CPAP pressure settings. It should be understood that this is meant to be exemplary only, and that other pressure support methodologies, including, but not limited to, BiPAP AutoSV, AVAPS, Auto CPAP, and BiPAP Auto, are within the scope of the present invention.

In the exemplary embodiment, patient interface device 14 includes a patient sealing assembly 28, which in the illustrated embodiment is a nasal mask. However, as noted elsewhere herein, other types of patient sealing assemblies, such as, without limitation, a nasal/oral mask, a nasal cushion, or a full face mask, which facilitate the delivery of the flow of breathing gas to the airway of a patient may be substituted for patient sealing assembly 28 while remaining within the scope of the present invention. Patient sealing assembly 28 includes a cushion 30 coupled to a frame member 32. In the illustrated embodiment, cushion 30 is defined from a unitary piece of soft, flexible, cushiony, elastomeric material, such as, without limitation, silicone, an appropriately soft thermoplastic elastomer, a closed cell foam, or any combination of such materials. Also in the illustrated embodiment, frame member 32 is made of a rigid or semi-rigid material, such as, without limitation, an injection molded thermoplastic or silicone, and includes a faceplate portion 34 to which cushion 30 is fluidly attached. A fluid coupling conduit 36 having an exhaust vent is coupled to an opening in faceplate portion 34 to allow the flow of breathing gas from pressure generating device base unit 4 to be communicated to an interior space defined by cushion 30, and then to the airway of a patient.

Frame member 32 also includes a forehead support member 38 that is coupled to the faceplate portion 34 by a connecting member 40. A forehead cushion 42 is coupled to the rear of forehead support 38 20. In the exemplary embodiment, forehead cushion 42 is made of a material that is similar to the material of cushion 30.

Patient interface device 14 also includes a headgear component 44 for securing patient interface device 14 to the head of patient 10. Headgear component 44 includes a back member 46, upper strap members 48 and lower strap members 50. In the exemplary embodiment, upper strap members 48 and lower strap members 50 each include a hook and loop fastening system, such as VELCRO®, provided on the end thereof to allow headgear component 44 to be secured in a known manner. It will be understood that the described hook and loop fastening arrangement is meant to be exemplary only, and that other selectively adjustable fastening arrangements are also possible within the scope of the present invention.

Furthermore, patient interface device 14 in the exemplary embodiment includes a degradation monitor 52 that is coupled to the frame member 32. As described in detail herein, degradation monitor 52 is structured to provide a human perceivable (e.g., visual, audio etc.) indication to patient 10 that alerts the patient when a determination has been made, as described herein, that a component of patient interface device 14, such as cushion 30, has degraded to a level such that performance of patient interface device 14 is compromised and such that the component should be replaced. Various embodiments of this degradation monitor concept are described in detail herein.

As described in detail herein, the degradation monitor concept of the present invention monitors degradation of a component of patient interface device 14 by measuring and monitoring a degradation metric that is indicative of the degradation over time of the component. A number of different degradation methods may be employed within the scope of the present invention. In the exemplary embodiment, the degradation metric is based on the amount of leak being experienced by patient interface device 14 (and may be a particular leak parameter such as large leak minutes (described below) or total leak total leak flow Qₗₑₐₖ), although it will be understood that the degradation metric may be based on any of a number of other parameters that indicate a change of performance of the patient interface device component over time such as, without limitation, time wearing patient interface device 14, therapy days missed (i.e., days in which patient interface device 14 and pressure support system 2 were not used for delivering therapy), therapy compliance over time, delivered flow rate over time, delivered pressure over time, or pressure variation over time.

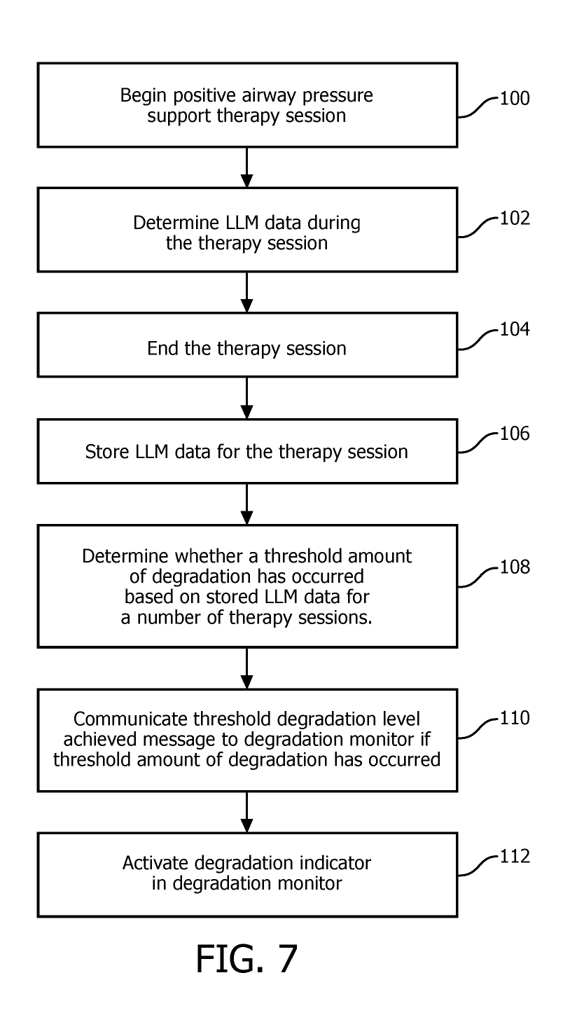

In particular exemplary embodiment described herein, the degradation metric is a parameter known as large leak minutes (LLM). As is known in the art, lm calculated by determining the total leak flow Q_{LEAK} during a therapy session and continuously comparing the total leak flow Qₗₑₐₖ to a predetermined large leak threshold value. The total cumulative time (in minutes) for which Qₗₑₐₖ exceeds the predetermined large leak threshold value is tracked and stored as the LLM parameter. Thus, for any desired time period (e.g., a day, a week, etc.), there will be a corresponding LLM value that has been determined. Furthermore, LLM may be averaged over a desired time such as a week or month, etc. The particular manner in which LLM is used to monitor degradation of patient interface device 14 according to various exemplary embodiments is described in detail elsewhere herein (FIGS. 6 and 7).

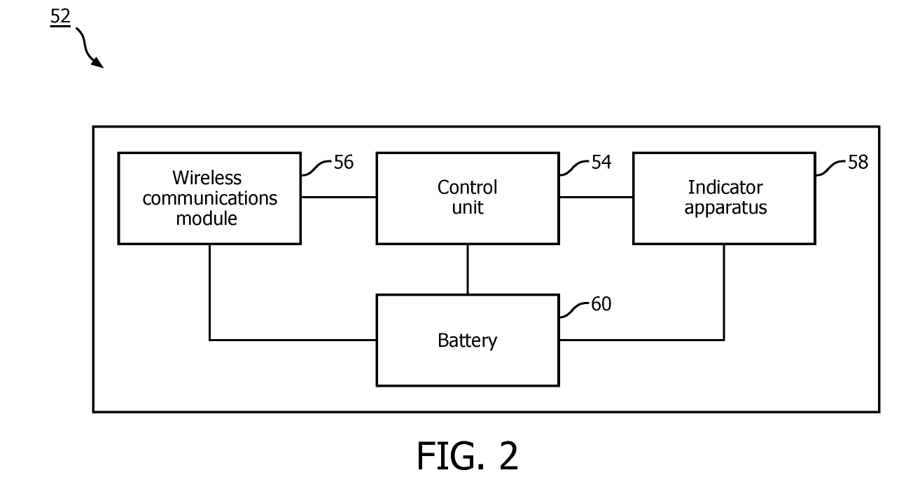

FIG. 2 is a block diagram of degradation monitor 52 according to the exemplary embodiment. As seen in FIG. 2, degradation monitor 52 includes a control unit 54. Control unit 54 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for controlling the operation of pressure support system 2. Degradation monitor 52 also includes a wireless communications module 56 that is structured to enable communication with short-range wireless communication module 26 of pressure generating device base unit 4. In the exemplary embodiment, wireless communications module 56 is a Bluetooth® module, although other types of wireless communication modules as described herein may also be used. Degradation monitor 52 further includes an electronic indicator apparatus 58 that is structured to enable degradation monitor 52 to provide a visual, audio or tactile indication to patient 10 when it is determined that a component of patient interface device 14 has degraded beyond a certain acceptable level (i.e., to a point where performance has been adversely affected).

For example, electronic indicator apparatus 58 may be a liquid crystal display (LCD), a number of colored light emitting diodes (LEDs), a speaker, or a vibrating module. Indicator apparatus 58 may also be a mechanically actuated device that can be actuated to provide tactile feedback, such as a pin or other feature that is caused to pop up and/or protrude from patient interface device 14 or a mechanism that emits a scent in order to provide the human perceptible indication. Finally, in the exemplary embodiment, degradation monitor 52 includes a battery 64 providing power to the components of degradation monitor 52. It will be understood, however, that this is meant to be exemplary only, and power may come from other sources, such as, without limitation, a solar cell, power generated from the leak of therapy air from patient interface device 14, power delivered from pressure generating device base unit 4 via a direct wired connection, or power delivered wirelessly via any suitable form of wireless charging (e.g., near field inductive coupling).

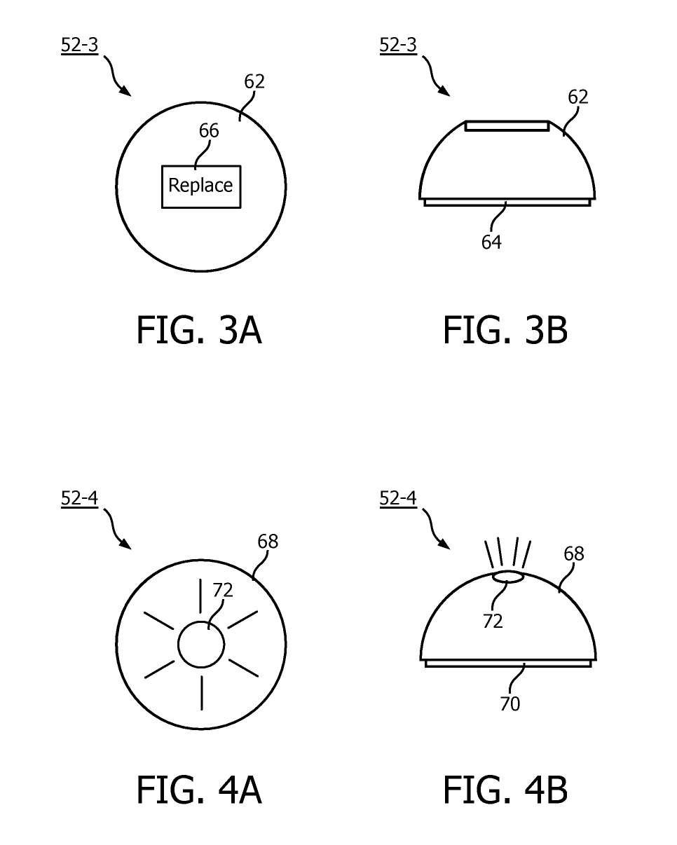

In the exemplary embodiment, degradation monitor 52 is structured to be selectively and removably coupled to patient interface device 14 (e.g., on frame member 32 or elsewhere) by, for example and without limitation, a suitable reusable adhesive or other reusable attachment mechanism such as a hook and loop fastening system like Velcro®. Examples of two such embodiments are shown in FIGS. 3A and 3B and FIGS. 4A and 4B. In particular, FIGS. 3A and 3B show degradation monitor 52-3 according to one particular exemplary embodiment that includes a housing 62, an attachment mechanism 64, such as a layer of reusable adhesive or reusable tape or a Velcro® pad, and indicator apparatus 58 in the form of LCD screen 66. In this embodiment, LCD screen 66 is structured to display a message such as “REPLACE” to the user when it has been determined that greater than a threshold amount of degradation has occurred. FIGS. 4A and 4B show degradation monitor 52-4 according to another particular exemplary embodiment that includes a housing 68, an attachment mechanism 70, such as a layer of adhesive or a Velcro® pad, and indicator apparatus 58 in the form of colored LED 72. In this embodiment, colored LCD 72 is structured to generate a light of a certain color (e.g., red) to the user when it has been determined that greater than a threshold amount of degradation has occurred. Colored LCD 72 may also be structured to generate light of a different color (e.g., green) prior to the time that degradation has occurred.

According to a further alternative embodiment, degradation monitor 52 may be assembled and fixedly and permanently attached to patient interface device 14 by a method such as over molding, sewing, riveting, attaching with a permanent adhesive, or any other suitable mechanism. In such an embodiment, degradation monitor 52 may be disposable.

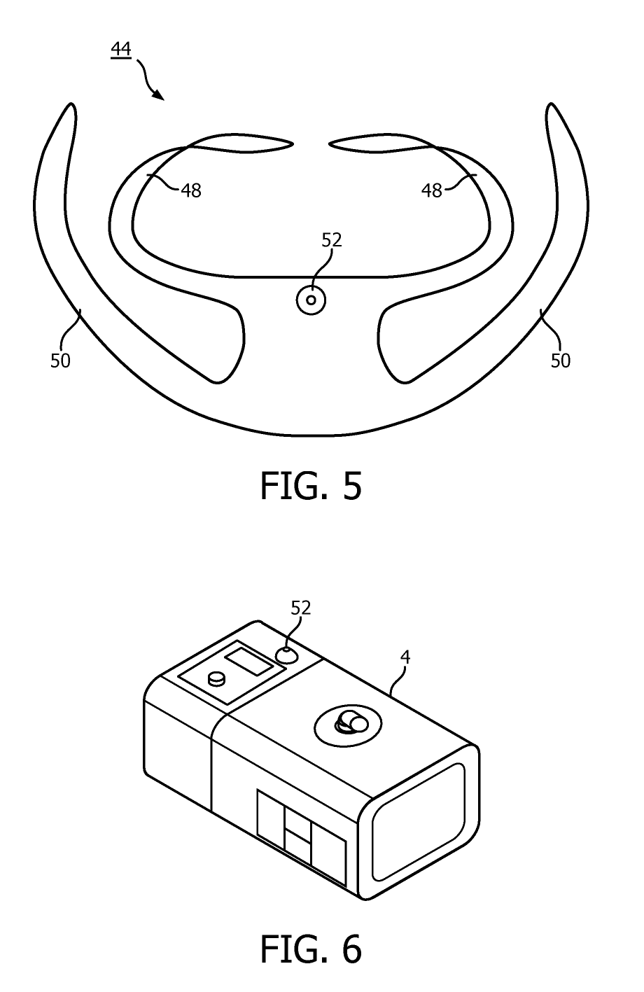

FIG. 5 is a schematic diagram of an alternative implementation of the disclosed concept. In particular, in this embodiment, degradation monitor 52 is coupled to a rear panel of headgear component 44 of patient interface device 14. It will be appreciated that degradation monitor 52 may also be coupled to other places, such as other portions of patient interface device 14 or the housing of pressure generating device base unit 4 as shown in FIG. 6, within the scope of the present invention.

FIG. 7 is a flowchart illustrating a method of operation of pressure support system 2 wherein degradation of a component (i.e., cushion 30) of patient interface device 14 is monitored according to one exemplary embodiment. As noted elsewhere herein, the method of operation shown in FIG. 7 employees LLM as a degradation metric. Referring to FIG. 7, the method begins at step 100, wherein positive airway pressure support therapy session using pressure support system 2 begins such that therapy air is delivered to patient 10 through patient interface device 14. Next, at step 102, LLM data is determined by controller 22 of pressure generating device base unit 4 during the therapy session as described herein. At step 104, the therapy session ends, and at step 106, the LLM data generated for the therapy session is stored by controller 22. Next, at step 108, controller 22 determines whether a threshold amount of degradation has occurred based on the stored LLM data for a number of sessions.

Step 108 may be performed in a number of different manners. In the exemplary embodiment, the LLM data for each of the therapy sessions conducted during a certain predetermined period, such as a week, is averaged and the calculated average LLM value for the period is compared to some predetermined threshold value. In the exemplary embodiment, the predetermined threshold value is determined in advance during a testing/learning phase and represents an LLM value for the particular cushion 30 that, if exceeded, will be considered to indicate that cushion 30 has degraded to a degree at which it needs to be replaced. In one example implementation, during the testing/learning phase, a baseline LLM value may be determined for the particular cushion that indicates acceptable performance. The threshold value may then be determined as some percentage above that value (e.g., 120%) that will be considered to indicate unacceptable degradation. In an alternative embodiment, the LLM data for each therapy session is, at the end of the therapy session, compared to a predetermined threshold value to determine whether a threshold amount of degradation has occurred. Still other examples of the manner in which step 108 may be performed are possible.

Next, at step 110, if it is determined in step 108 that the threshold amount of degradation has occurred, a message, referred to as a threshold degradation level achieved message, is communicated by short range wireless communications module 26 to wireless communications module 56 of degradation monitor 52. Then, at step 112, degradation monitor 52, in response to receiving the threshold degradation level achieved, will cause indicator apparatus 58 to be activated to provide an indication to patient 10 (e.g., visually as shown in FIGS. 3A and 4A, or audibly or tactilely) that cushion 30 of patient interface device 14 needs to be replaced. In response to receiving such an indication, patient 10 can replace cushion 30 and possibly other components of patient interface device 14 and seek reimbursement from their insurance company.

Thus, in the embodiment of FIG. 7, the storage of the degradation metric data and the determination as to when degradation has occurred is performed by pressure generating device base unit 4, with degradation monitor 52 functioning as a user interface. In a further alternative embodiment, the threshold degradation level achieved message may also (or instead) be sent to a handheld electronic device, such as a smart phone or a tablet computer, to enable that device to function as an additional (or alternate) user interface. In still a further alternative embodiment, the threshold degradation level achieved message may also be sent to a supplier of patient interface device 14 indicating to that supplier that a replacement component should be automatically shipped to the patient due to the level of degradation of the component currently being used. This communication may come directly from pressure generating device base unit 4 (in which case it would be provided with a wired or wireless communications capability to enable long-range data communications) or from the handheld electronic device just described.

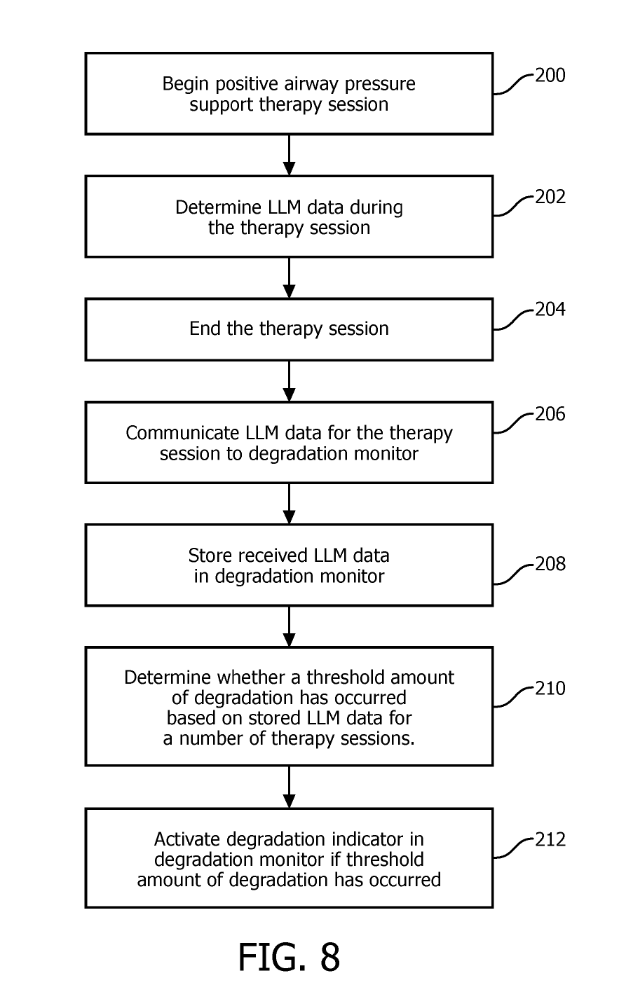

FIG. 8 is a flowchart of a method of operation of pressure support system 2 wherein degradation of a component (i.e., cushion 30) of patient interface device 14 is monitored according to another, alternative exemplary embodiment. In this alternative embodiment, pressure generating device base unit generates the degradation metric data, but it is degradation monitor 52 that stores the data and makes the determination as to when degradation has occurred.

In particular, referring to FIG. 8, the method begins at step 200, wherein a positive airway pressure support therapy session using pressure support system 2 begins such that therapy air is delivered to patient 10 through patient interface device 14. Next, at step 202, LLM data is determined by controller 22 of pressure generating device base unit 4 during the therapy session as described herein. At step 204, the therapy session ends, and at step 206, the LLM data generated for the therapy session is communicated from pressure generating device base unit for 2 degradation monitor 52 wirelessly as described herein. Then, at step 208, degradation monitor 52 stores the received LLM data and control unit 54. Next, at step 210, control unit 54 determines whether a threshold amount of degradation has occurred based on the stored LLM data for a number of therapy sessions. Step 210 may be performed as described in connection with step 108 of FIG. 6. At step 212, degradation monitor 52 will, in response to determining that a threshold amount of degradation has occurred in step 210, cause indicator apparatus 58 to be activated to provide an indication to patient 10 (e.g., visually as shown in FIGS. 3A and 4A, or audibly or tactilely) that cushion 30 of patient interface device 14 needs to be replaced. In response to receiving such an indication, patient 10 can replace cushion 30 and possibly other components of patient interface device 14 and seek reimbursement from their insurance company.

In the exemplary embodiments of FIGS. 7 and 8, the various steps therein are implemented in one or more routines comprising a plurality of instructions executable by controller 22 and control unit 54, as appropriate.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A pressure support system for delivering a flow of breathing gas to an airway of a patient, comprising:
a pressure generating base unit including a gas flow generator for generating the flow of breathing gas and a controller;
a patient interface device for delivering the flow of breathing gas to the airway of the patient; and
a degradation monitor apparatus separate and distinct from the pressure generating base unit, the degradation monitor apparatus including a control unit and an indicator apparatus, wherein the controller is structured to generate data indicative of a degradation metric based on use of the pressure support system and to communicate the data indicative of the degradation metric to the degradation monitor apparatus, and wherein the control unit of the degradation monitor apparatus is structured to cause the indicator apparatus to generate a human perceptible indication based on the data indicative of the degradation metric when the degradation metric indicates that a component of the patient interface device demonstrates at least a threshold level of degradation.

2. The pressure support system according to claim 1, wherein the controller is structured to generate first degradation metric data and to generate the data indicative of the degradation metric based on the first degradation metric data, the data indicative of the degradation metric being a threshold degradation level achieved message generated by the controller in response to a determination that the component of the patient interface device demonstrates at least the threshold level of degradation based on the first degradation metric data exceeding a predetermined threshold.

3. The pressure support system according to claim 2, wherein the controller is structured to generate second degradation metric data based on the first degradation metric data, wherein the first degradation data comprises a plurality of first degradation data values generated during a plurality first use periods, and wherein the second degradation metric data is a second degradation metric value based on each of the first degradation data values.

4. The pressure support system according to claim 1, wherein the data indicative of the degradation metric comprises first degradation metric data including a plurality of first degradation data values generated during a plurality first use periods, wherein the control unit of the degradation monitor apparatus is structured to generate second degradation metric data based on the first degradation metric data, wherein the second degradation metric data is a second degradation metric value based on each of the first degradation data values, and wherein the control unit of the degradation monitor apparatus is structured to cause the indicator apparatus to generate the human perceptible indication using the second degradation metric value responsive to the second degradation metric value indicating that the component of the patient interface device demonstrates at least the threshold level of degradation by exceeding a predetermined threshold.

5. The pressure support system according to claim 1, wherein the degradation metric is based on gas leaking from the patient interface device and not delivered to the airway of the patient.

6. A method of monitoring degradation of a component of a patient interface device of a pressure support system structured to deliver a flow of breathing gas to an airway of a patient, comprising:
generating data indicative of a degradation metric in a pressure generating base unit of the pressure support system based on use of the pressure support system;
communicating the data indicative of the degradation metric to a user interface apparatus having a control unit separate and distinct from the pressure generating base unit; and
generating from the user interface apparatus a human perceptible indication based on the data indicative of the degradation metric when the degradation metric indicates that the component of the patient interface device demonstrates at least a threshold level of degradation.

7. The method according to claim 6, wherein the user interface apparatus is a degradation monitor apparatus that is permanently affixed to the patient interface device or that is structured to be selectively and removably coupled to the patient interface device or the pressure generating device base unit.

8. The method according to claim 6, wherein the generating the data indicative of the degradation metric comprises generating first degradation metric data and generating the data indicative of the degradation metric based on the first degradation metric data, the data indicative of the degradation metric being a threshold degradation level achieved message generated in response to a determination that the component of the patient interface device demonstrates at least the threshold level of degradation based on the first degradation metric data exceeding a predetermined threshold.

9. The method according to claim 6, wherein the data indicative of the degradation metric comprises first degradation metric data including a plurality of first degradation data values generated during a plurality first use periods, further comprising generating second degradation metric data based on the first degradation metric data in the degradation monitor apparatus, wherein the second degradation metric data is a second degradation metric value based on each of the first degradation data values, and wherein the generating from the degradation monitor apparatus a human perceptible indication comprises generating the human perceptible indication using the second degradation metric value responsive to the second degradation metric value indicating that the component of the patient interface device demonstrates at least the threshold level of degradation by exceeding a predetermined threshold.

10. The method according to claim 6, wherein the degradation metric is based on gas leaking from the patient interface device and not delivered to the airway of the patient.

11. A degradation monitor apparatus for monitoring degradation of a component of a patient interface device of a pressure support system having a pressure generating base unit structured to deliver a flow of breathing gas to an airway of a patient, comprising:
a housing separate and distinct from the pressure generating base unit;
an indicator apparatus supported by the housing;
a wireless communications module within the housing; and
a control unit within the housing, wherein the control unit is structured to (i) receive data indicative of a degradation metric generated by the pressure support system and based on use of the pressure support system through the wireless communications module, and (ii) cause the indicator apparatus to generate a human perceptible indication based on the data indicative of the degradation metric when the degradation metric indicates that a component of the patient interface device demonstrates at least a threshold level of degradation.

12. The degradation monitor apparatus according to claim 11, wherein the data indicative of the degradation metric is a threshold degradation level achieved message generated by the pressure support system in response to a determination that the component of the patient interface device demonstrates at least the threshold level of degradation.

13. The degradation monitor apparatus according to claim 11, wherein the data indicative of the degradation metric comprises first degradation metric data including a plurality of first degradation data values generated by the pressure support system during a plurality first use periods, wherein the control unit is structured to generate second degradation metric data based on the first degradation metric data, wherein the second degradation metric data is a second degradation metric value based on each of the first degradation data values, and wherein the control unit is structured to cause the indicator apparatus to generate the human perceptible indication using the second degradation metric value responsive to the second degradation metric value indicating that the component of the patient interface device demonstrates at least the threshold level of degradation by exceeding a predetermined threshold.

14. The degradation monitor apparatus according to claim 11, wherein the degradation metric is based on gas leaking from the patient interface device and not delivered to the airway of the patient.

15. The degradation monitor apparatus according to claim 11, wherein the indicator apparatus includes at least one of an LCD display and one or more LEDs.
